(19) United States
(12) Patent Application Publication
(10) Pub. No.: US 2012/0029344 A1
(43) Pub. Date: Feb. 2, 2012

(54) RADIOLOGICAL IMAGE RADIOGRAPHIING METHOD AND APPARATUS

(75) Inventor: Hiroki NAKAYAMA, Kanagawa (JP)

(73) Assignee: FUJIFILM CORPORATION, Tokyo (JP)

(21) Appl. No.: 13/191,333

(22) Filed: Jul. 26, 2011

(30) Foreign Application Priority Data
Jul. 29, 2010 (JP) P2010-170624

(51) Int. Cl. A61B 6/02; A61B 8/00

(52) U.S. Cl. 600/427; 378/37; 378/41; 378/21; 378/62

(57) ABSTRACT

Provided is a technique capable of reducing the burden on an examinee and improving imaging efficiency. A radiographic method includes: radiating radiation to a subject in different radiographing directions by moving a radiation source; capturing radiological images in the different radiographing directions using the emission of radiation; and acquiring an image using a modality that acquires an image related to anatomical information of the subject which is different from the radiological images during the movement of the radiation source from a predetermined radiographing direction to the next radiographing direction.

### BACKGROUND OF THE INVENTION

1. Field of the Invention

The present invention relates to a radiological image radiographing method and apparatus that radiates radiation to a subject in a plurality of different radiographing directions by moving a radiation source and captures radiological images in the different radiographing directions using the radiation.

2. Description of the Related Art

An imaging method using radiation has been used in various kinds of fields. In particular, in the field of medical field, the imaging method using radiation is one of the most important means for diagnosis. A radiological image that is obtained by breast radiography for diagnosing a breast cancer is useful for finding a tumor and calcification, which are symptoms of a cancer. However, in some cases, it is difficult to find the tumor or calcification according to, for example, the mammary gland density of the examinee. Therefore, diagnosis made based on a radiological image and an ultrasonic image using both radiation and ultrasonic waves is being investigated. Radiography and ultrasonography have the following characteristics.

Radiography is suitable for finding calcification, which is an early symptom of the cancer, and can detect calcification with high resolution and high sensitivity. In particular, from women at the menopause, when mammary gland tissue starts to atrophy and is changed into a fatty material (a so-called “fat breast”), a large amount of information is obtained by radiography. However, radiography has the disadvantage in that it has a low capability of detecting tissue specificity (tissue characteristics).

The mammary gland has a uniform soft tissue density. Therefore, as in women from puberty to menopause, in the case of a breast in which the mammary gland is developed (a so-called “dense breast”), it is difficult to detect a tumor from the radiological image. In addition, in radiography, only a two-dimensional image of a subject being examined is obtained and projected onto the plane. Therefore, even though a tumor is found, it is difficult to understand information about the position or size of the tumor in the depth direction, for example.

In contrast, ultrasonography can detect the specificity of tissue (for example, the difference between a cystoma and a solid body) and can also detect a lobular cancer. In addition, in ultrasonography, it is possible to observe an image in real time and generate a three-dimensional image. However, in many cases, the accuracy of ultrasonography depends on the technique of the operator, such as a doctor, and the repeatability of the ultrasonography is low. In addition, it is difficult to observe fine calcification from the ultrasonic image.

As such, radiography and ultrasonography have advantages and disadvantages. Therefore, in order to reliably find a breast cancer, it is preferable to perform both radiography and ultrasonography. Radiography is performed with a subject (breast) being compressed by the compression plate. Therefore, in order to make a diagnosis based on the radiological image and the ultrasonic image of the subject in the same state, ultrasonography needs to be performed under the same conditions as those in which radiography is performed, that is, with the subject (breast) being compressed by the compression plate. Therefore, a medical imaging apparatus has been examined which uses both radiation and ultrasonic waves to capture the image of the mammary gland and the breast (for example, see JP1997-504211A (JP-H09-504211A)).

A technique has been known which combines a plurality of images and displays the combined image, thereby obtaining an image that can be stereoscopically viewed using parallax. The image that can be stereoscopically viewed (hereinafter, referred to as a stereoscopic image or a stereo image) is generated based on a plurality of images which are acquired by capturing the same subject in different directions and has parallax therebetween.

Moreover, such way of generating stereoscopic image is utilized not only in the field of digital cameras and televisions but also in the field of capturing a stereoscopic radiological image. That is, a test subject is irradiated with radiation from different directions. and then the radiation passing through the test subject is detected by a radiological image detector to acquire plural radiological images having parallax, and a stereoscopic image is generated based on the radiological images. By generating a stereoscopic image in this way, a radiological image with a sense of depth can be observed thereby enabling the observation of a radiological image more suitable for diagnosis.

### SUMMARY OF THE INVENTION

As described above, when both radiography and ultrasonography are used, radiography and ultrasonography need to be performed under the same conditions. Therefore, radiography and ultrasonography need to be performed with the breast being compressed by the compression plate.

The apparatus that captures the stereoscopic image needs to radiate radiation in different radiographing directions, thereby capturing a plurality of radiological images. Therefore, it is necessary to move the radiation source in order to capture each radiological image.

Therefore, when both the stereoscopic image and the ultrasonic image are captured. the time for which the breast is compressed increases and the burden on the examinee increases. In addition, the overall imaging time increases and imaging efficiency is reduced.

The present invention has been made in view of the above-mentioned problems and an object of the present invention is to provide a radiological image radiographing method and apparatus capable of reducing the burden on the examinee and improving imaging efficiency.

According to an aspect of the present invention, a radiological image radiographing method includes: radiating radiation to a subject in different radiographing directions by moving a radiation source; capturing radiological images in the different radiographing directions using the radiation; and acquiring an image related to anatomical information of the subject which is different from the radiological images using a modality during the movement of the radiation source from a predetermined radiographing direction to a next radiographing direction.

According to another aspect of the present invention, a radiological image radiographing apparatus includes: a radiation radiating unit that radiates to a subject in different radiographing directions by moving a radiation source; a radiological image detector that detects radiological images in the different radiographing directions which are captured by the emission of radiation by the radiation radiating unit; a modality that acquires an image related to anatomical information of the subject which is different from the radiological images; and a control unit that controls the modality to acquire the image related to the anatomical information of the subject which is different from the radiological images during the movement of the radiation source from a predetermined radiographing direction to the next radiographing direction.

In the radiological image radiographing apparatus according to the above-mentioned aspect of the present invention, the modality may acquire a tomographic image of the subject.

The modality may irradiates an ultrasonic wave to the subject to acquire an ultrasonic image of the subject.

The modality may include an ultrasonic probe that irradiates the ultrasonic wave to the subject and a scanning mechanism that performs scanning with the ultrasonic probe in a predetermined direction.

The radiological image radiographing apparatus according to the above-mentioned aspect may further include a scanning range determining unit that determines a scanning range of the ultrasonic probe by the scanning mechanism based on the radiological image captured by the emission of radiation to the subject in the predetermined radiographing direction.

The radiological image radiographing apparatus according to the above-mentioned aspect may further include a scanning range determining unit that receives an instruction to designate a predetermined range of the radiological image which is captured by the radiation to the subject in the predetermined radiographing direction and determines the range to be the scanning range of the ultrasonic probe by the scanning mechanism.

The scanning mechanism may perform scanning with the ultrasonic probe in the same direction as the moving direction of the radiation source.

The modality may acquire the tomographic image having a tomographic plane in a direction perpendicular to the moving direction of the radiation source.

The radiation radiating unit may radiates to the subject in the radiographing direction in which the radiological image forming a stereoscopic image is captured.

The subject may be a breast, and the radiological image detector may detect a breast image of the subject.

According to the radiological image radiographing method and apparatus of the present invention, during the movement of the radiation source from a predetermined radiographing direction to a next radiographing direction, the modality that acquires an image related to the anatomical information of the subject other than the radiological images is used to acquire the image related to the anatomical information of the subject which is different from the radiological images. Therefore, it is possible to reduce the overall imaging time and the burden on the examinee and improve imaging efficiency, as compared to, for example, a case in which the modality other than the radiological image radiographing apparatus is used to acquire an image before or after the plurality of radiological images is captured.

### BRIEF DESCRIPTION OF THE DRAWINGS

FIG. 1 is a diagram schematically illustrating the structure of a breast image capture and display system using a radiological image radiographing apparatus according to an embodiment of the present invention.

FIG. 2 is a diagram illustrating an also unit of the breast image capture and display system shown in FIG. 1, as viewed from the right direction of FIG. 1.

FIG. 3 is a diagram schematically illustrating the structure of a probe scanning mechanism.

FIG. 4 is a block diagram schematically illustrating the internal structure of a computer of the breast image capture and display system shown in FIG. 1.

FIG. 5 is a diagram illustrating a method of specifying a range in which mammary gland density is high.

FIG. 6 is a flowchart illustrating the operation of the breast image capture and display system using the radiological image radiographing apparatus according to the embodiment of the present invention.

### DESCRIPTION OF THE PREFERRED EMBODIMENTS

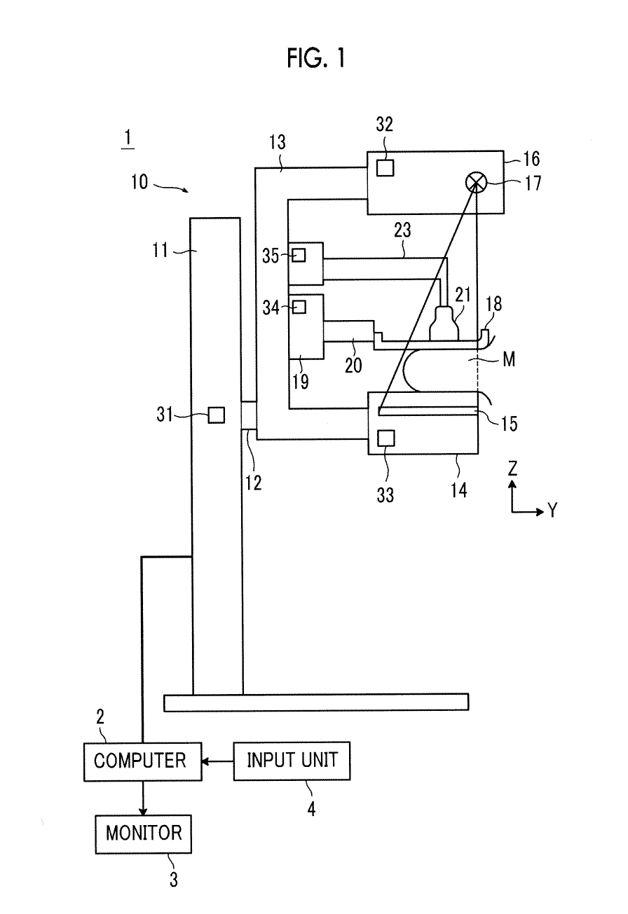

Hereinafter, a breast image capture and display system using a radiological image capture and display apparatus according to an embodiment of the present invention will be described with reference to the accompanying drawings. FIG. 1 is a diagram schematically illustrating the overall structure of the breast image capture and display system according to this embodiment.

As shown in FIG. 1, a breast image capture and display system 1 according to this embodiment includes a breast imaging apparatus 10, a computer 2 that is connected to the breast imaging apparatus 10, and a monitor 3 and an input unit 4 that are connected to the computer 2.

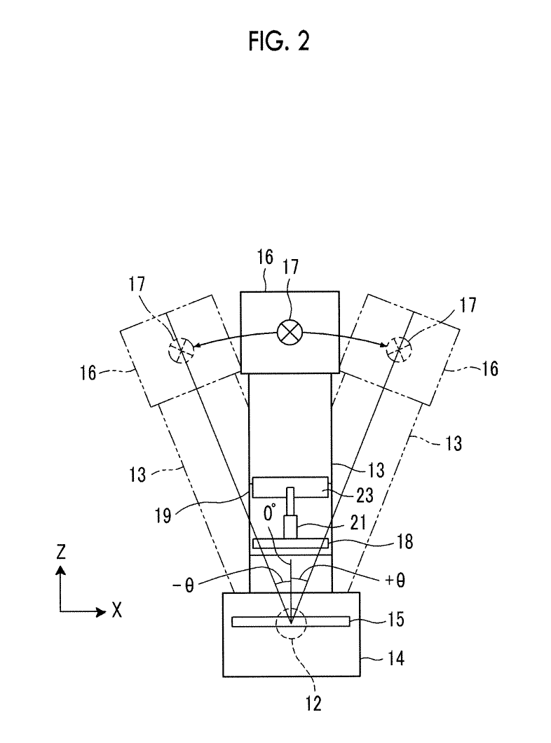

As shown in FIG. 1, the breast imaging apparatus 10 includes a base 11, a rotating shaft 12 that is movable in the vertical direction (Z direction) relative to the base 11 and is rotatable, and an arm unit 13 that is connected to the base 11 by the rotating shaft 12. FIG. 2 shows the arm unit 13, as viewed from the right direction of FIG. 1.

The arm unit 13 has a C-shape and includes one end to which a radiographic stand 14 is attached and the other end to which a radiation radiating unit 16 is attached so as to face the radiographic stand 14. The rotation and vertical movement of the arm unit 13 are controlled by an arm controller 31 that is incorporated into the base 11.

The radiographic stand 14 includes a radiological image detector 15, such as a flat panel detector, and a detector controller 33 that controls the reading of a charge signal from the radiological image detector 15. In addition, the radiographic stand 14 includes, for example, a circuit board provided with a charge amplifier that converts the charge signal read from the radiological image detector 15 into a voltage signal, a correlated double sampling circuit that samples the voltage signal output from the charge amplifier, and an A/D converter that converts the voltage signal into a digital signal.

The radiographic stand 14 is configured so as to be rotatable with respect to the arm unit 13. Therefore, even when the arm unit 13 is rotated with respect to the base 11, the direction of the radiographic stand 14 can be fixed with respect to the base 11.

The radiological image detector 15 can repeatedly record and read the radiological image and may he a so-called direct radiological image detector that directly receives radiation and generates charge or a so-called indirect radiological image detector that converts radiation into visible light and then converts the visible light into a charge signal. As a method of reading a radiological image signal, it is preferable to use a so-called TFT reading method of turning on or off a TFT (thin film transistor) switch to read the radiological image signal or a so-called optical reading method of radiating reading light to read the radiological image signal. However, the reading method is not limited thereto, and other methods may be used.

The radiation radiating unit 16 includes a radiation source 17 and a radiation source controller 32. The radiation source controller 32 controls the time when radiation is radiated from the radiation source 17 and the radiation generation conditions (for example, a tube current, time, and a tube current-time product) of the radiation source 17.

In addition, a compression plate 18 that is provided above the radiographic stand 14 and compresses the breast, a supporting portion 20 that supports the compression plate 18, and a moving mechanism 19 that moves the supporting portion 20 in the vertical direction (Z direction) are provided at the center of the arm unit 13. The position and compression pressure of the compression plate 18 are controlled by a compression plate controller 34.

The compression plate 18 is formed of an optically transparent member in order to check positioning and the compressed state when the breast is compressed. It is preferable that the compression plate 18 be made of a material that transmits radiation radiated from the radiation source 17 and easily transmits ultrasonic waves from an ultrasonic probe 21, which will be described below. The compression plate 18 may be made of, for example, a resin with appropriate acoustic impedance that affects the reflectance of ultrasonic waves and an appropriate attenuation coefficient that affects the attenuation of ultrasonic waves, such as polycarbonate, acryl, or polymethylpentene.

The ultrasonic probe 21 that moves along the upper surface of the compression plate 18 is supported by a probe scanning mechanism 23 on the compression plate 18.

The ultrasonic probe 21 includes a plurality of ultrasonic transducers which are one-dimensionally or two-dimensionally arranged. Each of the ultrasonic transducers transmits ultrasonic waves according to an applied driving signal, receives an ultrasonic echo, and outputs a received signal.

Each of the ultrasonic transducers is a transducer in which electrodes are formed at both ends of a piezoelectric material (piezoelectric body), such as piezoelectric ceramic whose representative example is PZT (Pb (lead) zirconate titanate) or a high-polymer piezoelectric element whose representative example is PVDF (polyvinylidene difluoride). When a pulse-shaped or a continuous-wave voltage is applied to the electrodes of the transducer. the piezoelectric body is expanded and contracted. Pulse-shaped or continuous-wave ultrasonic waves are generated from each transducer by the expansion and contraction and these ultrasonic waves are synthesized to form an ultrasonic wave beam. In addition, when receiving the propagated ultrasonic waves, each transducer is expanded and contracted and generates electric signals. These electric signals are output as the received signals of the ultrasonic waves and are then input to the computer 2 through a cable.

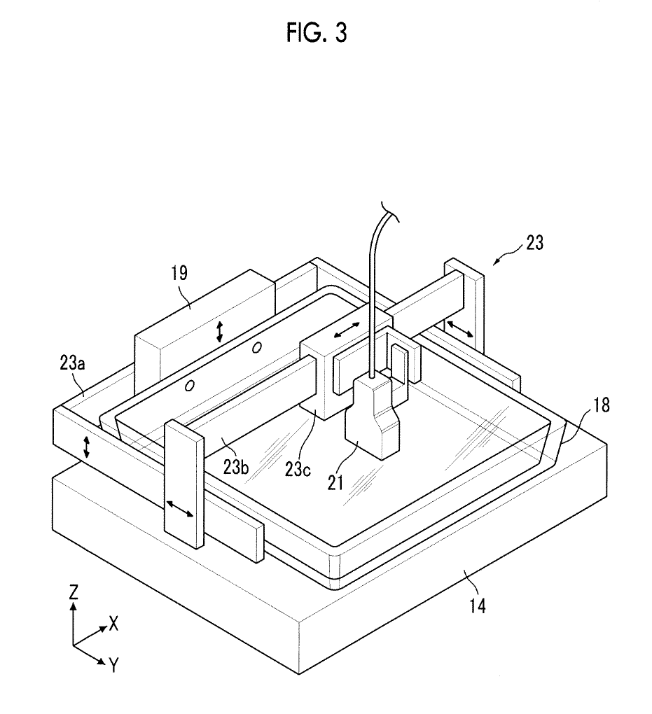

FIG. 3 is a diagram schematically illustrating the structure of the probe scanning mechanism 23. The probe scanning mechanism 23 includes a first moving member 23a that can move in the Z-axis direction, a second moving member 23b that can move in the Y-axis direction relative to the first moving member 23a, and a third moving member 23c that can move in the X-axis direction relative to the second moving member 23b. These moving members are driven by, for example, a stepping motor under the control of the probe controller 35 shown in FIG. 1.

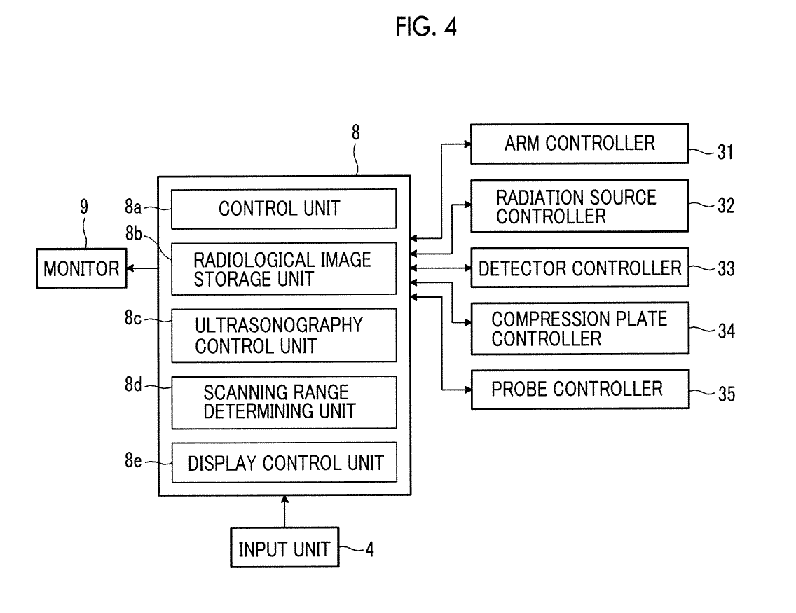

The computer 2 includes, for example, a central processing unit (CPU) and a storage device, such as a semiconductor memory, a hard disk, or an SSD. A control unit 8a, a radiological image storage unit 8b, an ultrasonography control unit 8c, a scanning range determining unit 8d, and a display control unit 8e shown in FIG. 4 are formed by these hardware components.

The control unit 8a outputs predetermined control signals to various kinds of controllers 31 to 35 to control the entire system. A detailed control method will be described below.

The radiological image storage unit 8b stores two radiological image signals acquired by the radiological image detector 15 in advance.

The ultrasonography control unit 8c includes, for example, a transmitting circuit, a receiving circuit, an A/D converter. and a signal processing unit.

The transmitting circuit generates a plurality of driving signals to be applied to the plurality of ultrasonic transducers based on a predetermined transmission delay pattern and supplies the driving signals to the ultrasonic probe 21.

The receiving circuit amplifies a plurality of received ultrasonic signals output from the plurality of ultrasonic transducers. The A/D converter converts analog received ultrasonic signals amplified by the receiving circuit into digital received ultrasonic wave signals.

The signal processing unit gives a delay time to each of the plurality of received ultrasonic wave signals based on a predetermined reception delay pattern and adds these received ultrasonic wave signals, thereby performing a reception focus process. Then, the signal processing unit performs a predetermined process to generate an ultrasonic image signal.

The scanning range determining unit 8d determines the scanning range of the ultrasonic probe 21 by the probe scanning mechanism 23 based on the radiological image signal that is acquired first, of the two radiological image signals stored in the radiological image storage unit 8b. Specifically, for example, since it is difficult to detect calcification in the range in which mammary gland density is high in the radiological image signal of the breast, it is effective to capture an image with the ultrasonic probe. Therefore, the scanning range determining unit 8d specifies the range in which the mammary gland density is high in the radiological image signal and determines the range to be the scanning range of the ultrasonic probe 21.

Specifically, for example, since a portion of the radiological image in which the mammary gland density is high is displayed in white, the scanning range determining unit 8d can specify the white portion, thereby specifying the region in which the mammary gland density is high. As a method of specifying the white portion, for example, the following method may be used: a method in which a radiological image is displayed on the monitor 3 based on the radiological image signal that is acquired first, the photographer uses the input unit 4 to select a white portion on the displayed radiological image, and the scanning range determining unit 8d acquires the information of the selected range: or a method in which the scanning range determining unit 8d recognizes an image based on the radiological image signal to automatically detect the white portion.

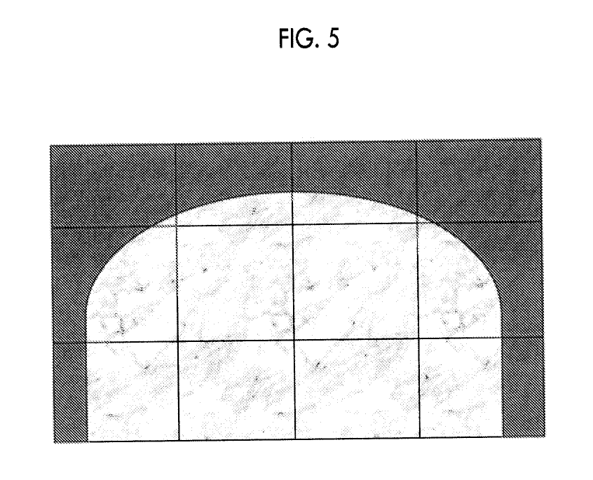

Specifically, for example, as shown in FIG. 5, the radiological image may be divided into a plurality of regions each having a predetermined size (in FIG. 5, 12 regions) and the photo may select a white region. Alternatively, the scanning range determining unit 8d may acquire the average value of the pixel value of each region and detect a (white) region with the smallest average value. In addition, in this case, the division (size) of the region may be determined based on, for example, the time for which scanning can be performed with the ultrasonic probe 21 or the size of the ultrasonic probe 21.

In this embodiment, as described above, the range in which the mammary gland density is high is determined to be the scanning range, but the present invention is not limited thereto. For example, other regions of interest, such as a region in which there is a large amount of calcification or a region in which there is a tumor mass, may be determined to be the scanning range.

As described above, the region of interest may not be necessarily automatically specified after the radiological image signal is analyzed. For example, the radiological image may be displayed on the monitor 3 based on the first radiological image signal and the observer may use the input unit 4 to designate a predetermined region of interest while viewing the displayed radiological image.

The display control unit 8e performs predetermined processing on two radiological image signals read from the radiological image storage unit 8b and displays the stereoscopic image of the breast on the monitor 3. In addition, the display control unit 8e performs predetermined processing on the ultrasonic image signal generated by the ultrasonography control unit 8c and displays the ultrasonic tomogaphic image of the breast on the monitor 3.

The input unit 4 is, for example, a keyboard or a pointing device, such as a mouse, and receives imaging conditions or an imaging start instruction input from the photographer.

The monitor 3 is configured such that it can display a stereoscopic image using two radiological image signals output from the computer 2 when the stereoscopic image is captured. As a structure that displays the stereoscopic image, for example, the following structure may be used in which two radiological images are respectively displayed on two screens based on two radiological image signals and, for example, a half mirror or a polarization glass is used such that one of the two radiological images is incident on the right eye of the observer and the other radiological image is incident on the left eye of the observer, thereby displaying a stereoscopic image. Alternatively, for example, the following structure may be used: a structure in which two radiological images are displayed so as to overlap each other with a positional deviation corresponding to a predetermined amount of parallax therebetween and a polarization glass is used to generate a stereoscopic image such that the observer can view the stereoscopic image; or a structure, such as a parallax barrier type or a lenticular type, in which two radiological images are displayed on a 3D display that can three-dimensionally display the radiological images, thereby generating a stereoscopic image.

A monitor that displays the stereoscopic image and a monitor that displays the ultrasonic image may be separately provided or a monitor common to the stereoscopic image and the ultrasonic image may be used.

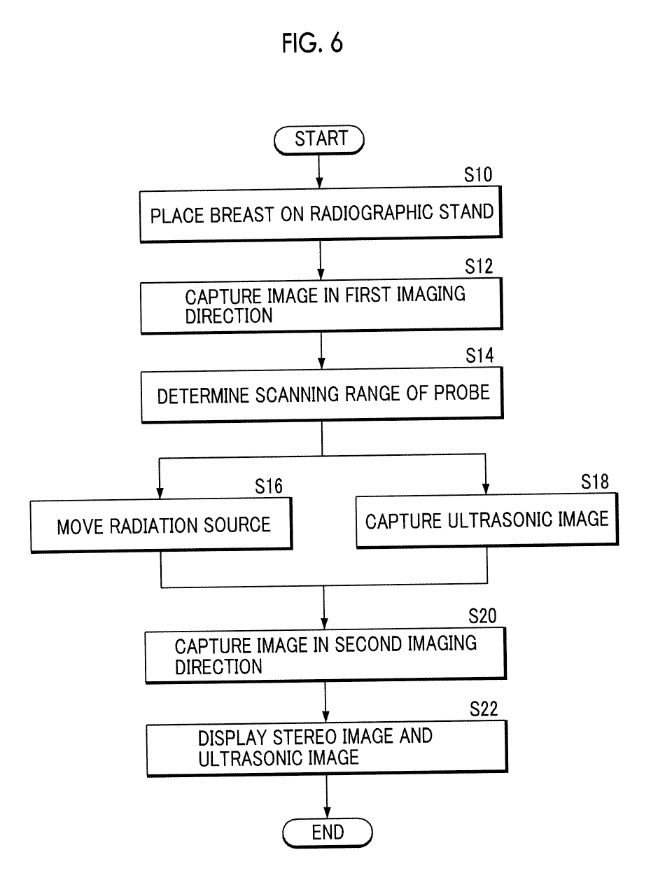

Next, the operation of the breast image capture and display system according to this embodiment will be described with reference to the flowchart shown in FIG. 6.

First, the breast M of the patient is placed on the radiographic stand 14 and the compression plate 18 compresses the breast M with a predetermined pressure (S10).

Then, the input unit 4 sequentially receives various kinds of imaging conditions and an image start instruction from the photographer.

When the input unit 4 receives the imaging start instruction, one of two radiological images forming the stereoscopic image of the breast M is captured (S12). Specifically, first, the control unit 8a reads the angle of convergence for capturing a predetermined stereoscopic image. In FIG. 2, the angle of convergence is two times more than the absolute value of θ. Then, the control unit 8a outputs the information of the read angle of convergence to the arm controller 31. In this embodiment, in this case, it is assumed that θ±2° is stored as the information of the angle of convergence in advance, but the present invention is not limited thereto. The photographer may use the input unit 4 to set an arbitrary angle of convergence.

The arm controller 31 receives the information of the angle of convergence output from the control unit 8a. Then, the arm controller 31 outputs a control signal based on the information of the angle of convergence such that the arm unit 13 rotates +θ° with respect to the direction vertical to the radiographic stand 14, as shown in FIG. 2. That is, in this embodiment, the arm controller 31 outputs a control signal such that the arm unit 13 rotates +2° with respect to the direction vertical to the radiographic stand 14.

The arm unit 13 rotates +2° in response to the control signal output from the arm controller 31. Then, the control unit 8a outputs control signals to the radiation source controller 32 and the detector controller 33 so as to perform the emission of radiation and the reading of the radiological image signal, respectively. In response to the control signals, the radiation source 17 radiates radiation, the radiological image detector 15 detects the radiological image of the breast captured in a +2° direction, and the detector controller 33 reads the radiological image signal. Then, predetermined signal processing is performed on the radiological image signal, and the radiological image signal is stored in the radiological image storage unit 8b of the computer 2. It is assumed that, when the radiological image is captured, the ultrasonic probe 21 is evacuated to the position where it does not affect image capture.

Then, one radiological image signal stored in the radiological image storage unit 8b is read and output to the scanning range determining unit 8d. Then, as described above, the scanning range determining unit 8d specifies the range in which the mammary gland density is high based on the input radiological image signal, determines the range to be the scanning range of the ultrasonic probe 21, and outputs the range to the probe controller 35 (S14).

Then, as shown in FIG. 2, the arm controller 31 returns the arm unit 13 to the initial position once and outputs a control signal such that the arm unit 13 rotates −θ° with respect to the direction vertical to the radiographic stand 14. That is, in this embodiment, the arm controller 31 outputs a control signal such that the arm unit 13 rotates −2° with respect to the direction vertical to the radiographic stand 14.

Then, the arm unit 13 rotates −2° in response to the control signal output from the arm controller 31. During the rotating operation, scanning is performed with the ultrasonic probe 21 and the ultrasonic image is captured (S16 and S18).

Specifically, the probe controller 35 drives the probe scanning mechanism 23 based on the input scanning range to scan the scanning range of the compression plate 18 with the ultrasonic probe 21.

In this embodiment, the ultrasonic probe 21 performs scanning in the X direction which is the moving direction of the radiation source 17. In this case, the ultrasonic probe 21 is arranged so as to capture a tomographic image having a tomographic plane in a direction perpendicular to the moving direction of the radiation source 17, that is, the Y direction.

The ultrasonography control unit 8c controls the ultrasonic probe 21 to transmit ultrasonic waves from each ultrasonic transducer of the ultrasonic probe 21 and receive the ultrasonic echo, and the ultrasonic probe 21 outputs the received signal to the ultrasonography control unit 8c. In the ultrasonography control unit 8c. a reception focus process is performed on the received signal, a predetermined process is performed on the received signal to generate an ultrasonic image signal, and the ultrasonic image signal is stored.

When the operation of rotating the arm unit 13 by −2° and the capture of he ultrasonic image end, a second radiological image is captured (S20).

Specifically, the control unit Ra outputs control signals to the radiation source controller 32 and the detector controller 33 to perform the emission of radiation and the reading of the radiological image, respectively. In response to the control signals, the radiation source 17 radiates radiation, the radiological image detector 15 detects the radiological image of the breast captured in a −2° direction. and the detector controller 33 reads the radiological image signal. Then, predetermined signal processing is performed on the radiological image signal, and the radiological image signal is stored in the radiological image storage unit 8b of the computer 2.

Then, two radiological image signals stored in the radiological image storage unit 8b are read and the ultrasonic image signal is read from the ultrasonography control unit 8c. Then, the read signals are input to the display control unit 8e and the display control unit 8e performs a predetermined process on the image signals and outputs the processed signals to the monitor 3. Then, the stereoscopic image and the ultrasonic tomographic image of the breast are displayed on the monitor 3 (S22).

In the above-described embodiment, the radiological image radiographing apparatus according to the present invention is applied to an apparatus for capturing a stereoscopic image, but the present invention is not limited thereto. The present invention can be applied to other imaging apparatuses that move the radiation source to radiates radiation to a subject in a plurality of different radiographing directions and capture radiological images in different radiographing directions using the emission of the radiation. For example, the present invention can be applied to a tomosynthesis system that generates the tomographic image of a subject using a plurality of radiological images obtained by capturing the subject in a plurality of radiographing directions as the imaging apparatus.

In the above-described embodiment, the ultrasonographic apparatus is used as the modality other than the imaging apparatus for capturing a radiological image, but the present invention is not limited thereto. Other kinds of modalities may be used. For example, an ultrasound modulated optical tomographic (UOT) apparatus using ultrasound modulated optical measurement may be used.

In the above description, the radiological image radiographing apparatus according to an embodiment of the present invention is applied to the breast image capture and display system, but the subject of the present invention is not limited to the breast. For example, the present invention can be applied to a radiological image capture and display system that captures the image of the chest or the head.

What is claimed is:

1. A radiological image radiographing method comprising:
radiating radiation to an subject in different radiographing directions by moving a radiation source:
capturing radiological images in the different radiographing directions using the radiating of radiation; and
acquiring an image related to anatomical information of the subject which is different from the radiological images using a modality during the movement of the radiation source from a predetermined radiographing direction to a next radiographing direction.

2. A radiological image radiographing apparatus comprising:
a radiation radiating unit that radiates radiation to a subject in different radiographing directions by moving a radiation source;
a radiological image detector that detects radiological images in the different radiographing directions which are captured by the radiation by the radiation radiating unit;
a modality that acquires an image related to anatomical information of the subject which is different from the radiological images; and
a control unit that controls the modality to acquire the image related to the anatomical information of the subject which is different from the radiological images during the movement of the radiation source from a predetermined radiographing direction to a next radiographing direction.

3. The radiological image radiographing apparatus according to claim 2,
wherein the modality acquires a tomographic image of the subject.

4. The radiological image radiographing apparatus according to claim 3,
wherein the modality irradiates an ultrasonic wave to the subject to acquire an ultrasonic image of the subject.

5. The radiological image radiographing apparatus according to claim 4,
wherein the modality comprises:
an ultrasonic probe that irradiates the ultrasonic wave to the subject; and
a scanning mechanism that performs scanning with the ultrasonic probe in a predetermined direction.

6. The radiological image radiographing apparatus according to claim 5, further comprising:
a scanning range determining unit that determines a scanning range of the ultrasonic probe by the scanning mechanism based on the radiological image captured by the radiation to the subject in the predetermined radiographing direction.

7. The radiological image radiographing apparatus according to claim 5, further comprising:
a scanning range determining unit that receives an instruction to designate a predetermined range of the radiological image which is captured by the radiation to the subject in the predetermined radiographing direction and determines the range to be the scanning range of the ultrasonic probe by the scanning mechanism.

8. The radiological image radiographing apparatus according to claim 5,
wherein the scanning mechanism performs scanning with the ultrasonic probe in the same direction as the moving direction of the radiation source.

9. The radiological image radiographing apparatus according to claim 6,
wherein the scanning mechanism performs scanning with the ultrasonic probe in the same direction as the moving direction of the radiation source.

10. The radiological image radiographing apparatus according to claim 7,
wherein the scanning mechanism performs scanning with the ultrasonic probe in the same direction as the moving direction of the radiation source.

11. The radiological image radiographing apparatus according to claim 3,
wherein the modality acquires the tomographic image having a tomographic plane in a direction perpendicular to the moving direction of the radiation source.

12. The radiological image radiographing apparatus according to claim 4,
wherein the modality acquires the tomographic image having a tomographic plane in a direction perpendicular to the moving direction of the radiation source.

13. The radiological image radiographing apparatus according to claim 6,
wherein the modality acquires the tomographic image having a tomographic plane in a direction perpendicular to the moving direction of the radiation source.

14. The radiological image radiographing apparatus according to claim 7,
wherein the modality acquires the tomographic image having a tomographic plane in a direction perpendicular to the moving direction of the radiation source.

15. The radiological image radiographing apparatus according to claim 2,
wherein the radiation radiating unit radiates radiation to the subject in the radiographing direction in which the radiological image forming a stereoscopic image is captured.

16. The radiological image radiographing apparatus according to claim 4,
wherein the radiation radiating unit radiates radiation to the subject in the radiographing direction in which the radiographing image forming a stereoscopic image is captured.

17. The radiological image radiographing apparatus according to claim 6,
wherein the radiation radiating unit radiates radiation to the subject in the radiographing direction in which the radiological image forming a stereoscopic image is captured.

18. The radiological image radiographing apparatus according to claim 2,
wherein the subject is a breast, and
the radiological image detector detects a breast image of the subject.

19. The radiological image radiographing apparatus according to claim 4,
wherein the subject is a breast, and
the radiological image detector detects a breast image of the subject.

20. The radiological image radiographing radiographic apparatus according to claim 6,
wherein the subject is a breast, and
the radiological image detector detects a breast image of the subject.
